(19) United States
(12) Patent Application Publication
(10) Pub. No.: US 2023/0151387 A1
(43) Pub. Date: May 18, 2023

(54) COVID-19 VACCINE BASED ON THE MYXOMA VIRUS PLATFORM

(71) Applicant: ARIZONA BOARD OF REGENTS ON BEHALF OF ARIZONA STATE UNIVERSITY, Scottsdale, AZ (US)

(72) Inventors: Grant MCFADDEN, Tempe, AZ (US); Mohammed Masmudur RAHMAN, Chandler, AZ (US); Brenda G. HOGUE, Tempe, AZ (US)

(21) Appl. No.: 17/916,788

(22) PCT Filed: Apr. 2, 2021
(86) PCT No.: PCT/US2021/025535

§ 371 Date: Oct. 3, 2022

Related U.S. Application Data
(60) Provisional application No. 63/004,072, filed on Apr. 2, 2020.

(51) Int. Cl. C12N 15/86; C07K 14/005; A61K 39/215; A61P 31/14

(52) U.S. Cl. CPC C12N 15/86; C07K 14/005; A61K 39/215; A61P 31/14; C12N 2770/20022; C12N 2770/20034; C12N 2770/20052; C12N 2710/24043; A61K 2039/5256

(57) ABSTRACT

The present invention provides myxoma viral vectors that encode severe acute respiratory syndrome coronavirus 2 antigens and that can facilitate expression and secretion of virus-like particles (VLPs). Also provided are methods of making said VLPs in mammalian cells and using said VLPs and myxoma viral vectors to induce an immune response in a subject.

### CROSS-REFERENCE TO RELATED APPLICATIONS

This application claims priority to U.S. Provisional Application No. 63/004,072 filed on Apr. 2, 2020, the contents of which are incorporated by reference in their entireties.

### SEQUENCE LISTING

A Sequence Listing accompanies this application and is submitted as an ASCII text file of the sequence listing named “112624_01246_ST25.txt” which is 48.9 KB in size and was created on Mar. 24, 2021. The sequence listing is electronically submitted via EFS-Web with the application and is incorporated herein by reference in its entirety.

### BACKGROUND

Coronaviruses (CoV) constitute a large family of positive-stranded, enveloped RNA viruses that infect a broad range of mammalian and avian species. The viruses cause primarily respiratory and enteric diseases. In the last two decades three new zoonotic CoVs have emerged to infect humans. The most recent emergence of SARS-CoV-2 that continues to spread across China and globally raises many scientific and public health questions and challenges. Development of effective vaccines and antiviral therapeutics and rapidly deployment of both is a pressing need. This will be an even more critical priority if SARS-CoV-2 continues to spread and becomes endemic in the respiratory virus disease landscape. Previous work with the other two recent emergent pathogenic human CoVs, severe acute respiratory syndrome (SARS-CoV) and Middle East respiratory syndrome (MERS-CoV), provides insight and platforms that can help expedite the process, but none of these have moved beyond early trial stages. Much remains to be learned about the SARS-CoV-2 and its interplay with its human host and what will constitute the most effective, safe vaccine strategy. There are currently seven CoVs that infect humans, HCoVs 0C43, 229E, NL63 and HKU1, that cause seasonal upper respiratory infections, in addition to the three more pathogenic viruses. The human viruses are thought to have emerged from zoonotic hosts to infect humans. Viral genomic analyses indicate that the human viruses are related to bat CoVs. A large number of novel CoVs have been identified in bat populations since identification of SARS-CoV and the expectation is that we will continue to have spillover of these viruses to humans. This reinforces the need for development of vaccines against emergent CoVs.

### SUMMARY OF THE INVENTION

In a first aspect, provided herein is a myxoma virus (MYXV) comprising a polynucleotide encoding a Severe Acute Respiratory Syndrome Coronavirus 2 (SARS-CoV-2) antigen selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, SARS-CoV-2 nucleocapsid (N) protein (SEQ ID NO:5) or a sequence at least 95% identical thereto, and combinations thereof.

In some embodiments, the MYXV comprises one or more polynucleotides encoding at least two SARS-CoV-2 antigens selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, and SARS-CoV-2 nucleocapsid (N) protein (SEQ ID NO:5) or a sequence at least 95% identical thereto.

In some embodiments, the MYXV comprises one or more polynucleotides encoding at least three SARS-CoV-2 antigens selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, and SARS-CoV-2 nucleocapsid (N) protein (SEQ ID NO:5) or a sequence at least 95% identical thereto.

In some embodiments, the MYXV comprises one or more polynucleotides encoding at least four SARS-CoV-2 antigens selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, and SARS-CoV-2 nucleocapsid (N) protein (SEQ ID NO:5) or a sequence at least 95% identical thereto.

In some embodiments, the MYXV comprises a modification at or adjacent to one or more genes associated with rabbit cell tropism. In some embodiments, the one or more genes associated with rabbit cell tropism are selected from the group consisting of M11L, M063, M135R, M136R, M153, M154, M-T2, M-T4, M-T5, and M-T7. In some embodiments, the MYXV comprises a partial or full deletion of the M153 and M154 genes. In some embodiments, the MYXV comprises a modification of the M153 and M154 genes that impairs the expression of the M153 and M154 genes. In some embodiments, the polynucleotide encoding the SARS-CoV-2 antigen replaces the M153 or the M154 gene in the MYXV genome. In some embodiments, the polynucleotide encoding the SARS-CoV-2 antigen is inserted between the M153 gene and the M154 gene within the MYXV genome.

In a second aspect, provided herein is a method for producing a SARS-CoV-2 virus-like particle (VLP) comprising transfecting a mammalian cell with a MYXV as described herein and extracting the SARS-CoV-2 VLP from the mammalian cell. In some embodiments, the mammalian cell is selected from the group consisting of Chinese Hamster Ovary (CHO), Madin-Darby Canine Kidney (MDCK), Vero, and HEK 293T.

In a third aspect, provided herein is a vaccine composition comprising a MYXV as described herein and a pharmaceutically acceptable carrier. In some embodiments the composition additionally comprises an adjuvant.

In a forth aspect, provided herein is a method for inducing an immune response in a subject comprising administering an effective amount of a vaccine composition described herein to the subject. In some embodiments, the subject is human. In some embodiments, the vaccine composition is administered systemically. In some embodiments, the vaccine composition is administered by injection. In some embodiments, the vaccine is administered intranasally. In some embodiments, the vaccine composition is administered in at least two doses.

In a fifth aspect, provided herein is VLP produced by the methods described herein.

In a sixth aspect, provided herein is a method of inducing an immune response in a subject comprising administering an effective amount of a VLP produced by the methods described herein to the subject.

### BRIEF DESCRIPTION OF DRAWINGS

FIGS. 1A-1C show schematic representations of recombinant myxoma viruses expressing various SARS-CoV-2 proteins to make virus-like particles (VLPS). (1A) Schematic representation of the recombinant myxoma viruses expressing SARS-CoV-2 spike (S) protein. (1B) Schematic representation of the recombinant myxoma viruses expressing SARS-CoV-2 envelope (E), membrane (M), and nucleocapsid (N) proteins. (1C) Schematic representation of the recombinant myxoma viruses expressing SARS-CoV-2 spike (S), envelope (E), membrane (M), and nucleocapsid (N) proteins.

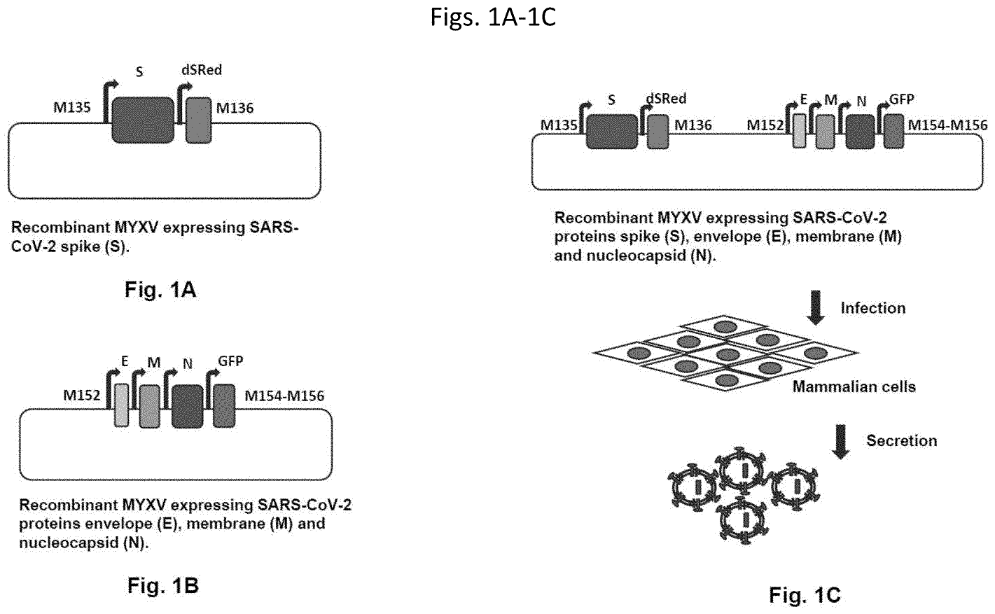

FIGS. 2A-2B depict the generation of vMyx-SARS2-S-dsRed. (2A) Schematic representation of the generation of recombinant MYXV, vMyx-SARS2-S-dsRed expressing SARS-CoV-2 spike (S) protein. (2B) RK13 cells were infected with wild-type MYXV, vMyx-dsRed and vMyx-SARS2-S-dsRed at a multiplicity of infection (MOI) of 0.01 for 48 h and single foci images were taken using fluorescence microscope. Single foci images of wild-type vMyx-dsRed (MYXV expressing only dsRed) are shown as a control.

FIGS. 3A-3B depict the generation of vMyx-SARS2-E-M-N-GFP. (3A) Schematic representation of the generation of recombinant MYXV, vMyx-SARS2-E-M-N-GFP expressing SARS-CoV-2 envelope (E), membrane (M), and nucleocapsid (N) proteins. (3B) RK13 cells were infected with wild-type MYXV, vMyx-GFP and vMyx-SARS2-E-M-N-GFP at a multiplicity of infection (MOI) of 0.01 for 48 h and single foci images were taken using fluorescence microscope. Single foci images of wild-type vMyx-GFP (MYXV expressing only GFP) are shown as a control.

FIGS. 4A-4B depict the generation of vMyx-SARS2-S-E-M-N-dsRed-GFP. (4A) Schematic representation of the generation of recombinant MYXV, vMyx-SARS2-S-E-M-N-dsRed-GFP expressing SARS-CoV-2 spike (S), envelope (E), membrane (M), and nucleocapsid (N) proteins. (4B) RK13 cells were infected with purified vMyx-SARS2-S-E-M-N-dsRed-GFP at a multiplicity of infection (MOI) of 0.01 for 48 h and single foci images were taken using fluorescence microscope.

FIG. 5 shows a schematic representation of the generation of recombinant MYXV, vMyx-SARS2-Hexa Pro S-dsRed expressing SARS-CoV-2 modified spike (S) protein Hexa Pro S.

FIG. 6 shows a schematic representation of the generation of recombinant MYXV, vMyx-SARS2-Hexa Pro S-E-M-N-dsRed-GFP expressing SARS-CoV-2 spike (Hexa Pro S), envelope (E), membrane (M), and nucleocapsid (N) proteins.

### DETAILED DESCRIPTION OF THE INVENTION

The present disclosure describes severe acute respiratory syndrome coronavirus 2 (SARS-CoV-2) vaccine compositions, as well as methods for making and using the same.

Severe acute respiratory syndrome coronavirus 2 (SARS-CoV-2) is the third highly pathogenic human CoV to emerge in the past two decades. The virus causes COVID-19, a severe respiratory disease with an estimated mortality of 2-3% that rapidly spread across China beginning in late 2019. As of April 2021, the virus caused greater than 129,000,000 cases and greater than 2,800,000 deaths worldwide, with continuing spread. The virus has affected 219 countries and territories during this period. Like other CoVs, the spike (S) protein is assumed spread globally to be the major target for neutralizing antibodies. SARS-CoV-2 S protein binds to the receptor, angiotensin-converting enzyme 2(ACE2), through its receptor binding domain (RBD). The RBDs for other CoVs are immunogenic and a major neutralizing determinant. Significant research has been directed toward vaccine development for the other recently emerged human pathogenic CoVs, severe acute respiratory syndrome (SARS) and Middle East respiratory syndrome (MERS), but none is yet available. Nonetheless, earlier studies should help expedite progress toward SARS-CoV-2 development. Much remains to be learned about SARS-CoV-2. There are significant concerns about the continuing spread of the virus and the possibility that it will become embedded in the viral respiratory disease landscape that will be encountered seasonally. Thus, development of a safe and effective vaccine against the virus is a significant priority. The long-term goal of this project is to develop a safe, efficacious vaccine(s) against SARS-CoV-2. Standard molecular biology, biochemical approaches, vaccination and immunogenicity assessment will be used to generate virus-like-particles (VLPS) in mammalian cells. The goal of this work is to optimally produce VLPs in mammalian cells and evaluate immune responses elicited in mice vaccinated with the VLPS.

SARS-CoV-2 includes membrane (M), spike (S), envelope (E), and nucleocapsid (N) structural proteins. The M, S, and E proteins provide the structure of the exterior viral envelope. The S protein is a glycoprotein that mediates receptor binding and fusion during entry into a host cell. The N protein is an internal structural component that encapsulates the SARS-CoV-2 viral genome. In some embodiments, the SARS-CoV-2 protein(s) used with the present invention have the amino acid sequences found in the original L strain of the virus that appeared in Wuhan in December 2019. Thus, in some embodiments, the S protein of SARS-CoV-2 has the sequence of SEQ ID NO:1, the receptor binding domain (RBD) of the S protein has the sequence of SEQ ID NO:2 (=amino acids 318-510 of SEQ ID NO:1), the M protein has the sequence of SEQ ID NO:3, the E protein has the sequence of SEQ ID NO:4, and/or the N protein has the sequence of SEQ ID NO:5.

In some embodiments, the S protein used with the present invention has been modified to improve its expression from the viruses described herein. For example, the inventors have generated a modified S protein termed “Hexa Pro S” (SEQ ID NO:14) that includes a mutation in the furin cleavage site (rrar>gsas) and six proline mutations (i.e., F817P, A892P, A899P, A942P, KV986/7>PP) that collectively stabilize the pre-fusion S protein. Thus, in some embodiments, the S protein has the sequence of SEQ ID NO:14.

In some embodiments, the DNA sequence(s) encoding the SARS-CoV-2 protein(s) used with the present invention are codon-optimized for expression in mammalian cells. For example, in some embodiments, the S protein is encoded by SEQ ID NO:18, the E protein is encoded by SEQ ID NO:19, the M protein is encoded by SEQ ID NO:20, and/or the N protein is encoded by SEQ ID NO:21.

As used herein, “SARS-CoV-2 antigen” refers to a SARS-CoV-2 protein, a sequence at least 90% identical thereto, a fragment thereof, or combinations thereof that may be used to elicit an immune response in a subject. The SARS-CoV-2 antigen may be the SARS-CoV-2 S protein, the SARS-CoV-2 M protein, the SARS-CoV-2 E protein, the SARS-CoV-2 N protein, the SARS-CoV-2 S protein RBD, a protein with a sequence having at least 90%, 95%, 98%, or 99% sequence identity thereto, or combinations thereof.

As used herein, the phrases “% sequence identity,” “percent identity,” or “% identity” are used interchangeably and refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. A suite of commonly used and freely available sequence comparison algorithms is provided by the National Center for Biotechnology Information (NCBI) Basic Local Alignment Search Tool (BLAST® alignment tool), which is available from several sources, including the NCBI, Bethesda, Md., at its website. The BLAST® alignment tool software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Polynucleotides encoding any of the SARS-CoV-2 antigens described herein are provided. As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

Polynucleotides homologous to the polynucleotides described herein are also provided. Those of skill in the art understand the degeneracy of the genetic code and that a variety of polynucleotides can encode the same polypeptide. In some embodiments, the polynucleotides (i.e., polynucleotides encoding the SARS-CoV-2 antigens described herein) may be codon-optimized for expression in a particular cell including, without limitation, a plant cell, mammalian cell, insect cell, bacterial cell, or fungal cell. While particular polynucleotide sequences are disclosed herein, any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

In another aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. As used herein, a polynucleotide is “operably connected” or “operably linked” when it is placed into a functional relationship with a second polynucleotide sequence.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease Si), as well as protein binding domains (consensus sequences) responsible for the binding of RNA polymerase.

Heterologous promoters useful in the practice of the present invention include, but are not limited to, constitutive, inducible, temporally regulated, developmentally regulated, chemically regulated, tissue-preferred and tissue-specific promoters. The heterologous promoter may be a plant, animal, bacterial, fungal, viral, or synthetic promoter. Suitable promoters are known and described in the art. Suitable promoters for expression in plants include, without limitation, the 35S promoter of the cauliflower mosaic virus, ubiquitin, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter.

In some embodiments, the promoter is viral synthetic late promoter (SLP). In some embodiments, the SLP has the sequence 5′-CATATTGAAGAGACAGAGTGATATAT-3′ (SEQ ID NO:10). Those of skill in the art are familiar with a wide variety of additional promoters for use in various cell types.

The constructs provided herein may include a translation enhancing element (TEE) operably linked to any one of the polynucleotides described herein.

As used herein “translation enhancing elements (TEE),” refers to polynucleotide sequences that mediate cap-independent translation initiation. A TEE polynucleotide refers to both the RNA polynucleotide being translated and the DNA polynucleotide encoding said RNA polynucleotide. Identification of TEEs is described in US Publication No. 20130230884 and described by Wellensiek et al. (“Genome-wide profiling of cap-independent translation enhancing elements in the human genome,” Nat Methods, 2013, 10(8):747-750). Suitable TEEs are also described in US Publication No. 20140255990 and Wellensiek et al. (“A leader sequence capable of enhancing RNA expression and protein synthesis in mammalian cells,” Protein Sci., 2013, 22(10):1392-1398). In some embodiments, the TEE includes the sequence 5′-AAAACTGCTAA-3′ (SEQ ID NO:6). In some embodiments, the TEE includes the sequence 5′-CATATTGAAGAGACAGAGTGATATATAAAACTGCTAA-3′ (SEQ ID NO:7). In some embodiments, the TEE includes the sequence 5′-AGAACCATATTGAAGAGACAGAGTGATATATAAAACTGCTAA-3′ (SEQ ID NO:8). In some embodiments, the TEE includes the sequence 5′-AGAACCATATTGAAGAGACAGAGTGATATATAAAACTGCTAACTCAA GCAGCACAAGAATTAAATGAATACCAAGAAAATACTTGGCCAG-3′ (SEQ ID NO:9). In some embodiments, a polynucleotide sequence may act as both a promoter and a TEE.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Viral genomes are also included as vectors, including vectors based on viral genomes. Vectors may carry genetic elements, such as those that confer resistance to certain drugs or chemicals.

In some embodiments, the vector is a myxoma viral vector (MYXV). In some embodiments, the engineered myxoma virus includes a polynucleotide encoding one or more SARS-CoV-2 antigens. Suitable MYXV vectors are disclosed in WO 2020014670, which is incorporated herein by reference. Myxoma virus is a rabbit pathogen, but does not cause disease in humans. Thus, it may be an effective viral vector for human vaccines.

The MYXV may include a modification at or adjacent to one or more genes associated with rabbit cell tropism. In some instances, the one or more genes associated with rabbit cell tropism comprises M11L, M063, M135R, M136R, M153, M154 M-T2, M-T4, M-T5, M-T7, or combinations thereof. In some instances, the one or more genes associated with rabbit cell tropism comprise M135R, M136R, or a combination thereof. In some embodiments, the modification is a deletion that impairs the function of a protein encoded by the M135R gene. In some embodiments, the modification is a partial deletion (e.g., a 10%, 20%, 30%, 40%, 50%, 60%, 70%, 80%, 90%, or 95% deletion) of the M135R gene. In some embodiments, the modification is a full deletion of the M135R gene. In some embodiments, the modification is a replacement of the M135R gene with the polynucleotide encoding the SARS-CoV-2 antigen.

In some embodiments, the MYXV includes a polynucleotide encoding a SAR-CoV-2 antigen inserted between M135 gene and M136 gene within the MYXV genome. In some embodiments, the MYXV includes a polynucleotide encoding a SAR-CoV-2 antigen inserted between M152 gene and M154 gene within the MYXV genome.

In some instances, the one or more genes associated with rabbit cell tropism comprise M153, M154, or a combination thereof. In some embodiments, the modification is a deletion that impairs the function of a protein encoded by the M153 and/or M154 genes. In some embodiments, the modification is a partial deletion (e.g., a 10%, 20%, 30%, 40%, 50%, 60%, 70%, 80%, 90%, or 95% deletion) of the M153 and/or M154 genes. In some embodiments, the modification is a full deletion of the M153 and/or M154 genes. In some embodiments, the modification is a replacement of the M153 and/or M154 genes with the polynucleotide encoding the SARS-CoV-2 antigen. In some embodiments, the MYXV includes a polynucleotide encoding a SAR-CoV-2 antigen inserted between M153 gene and M154 gene within the MYXV genome.

In some aspects, provided herein are virus-like particles (VLPs) incorporating the SARS-CoV-2 antigens described herein. As used herein, “virus-like particles (VLPs)” refers to particles that include one or more viral proteins and mimics the structure of the native virus but lack the viral genome. In some embodiments, the VLP includes the S protein. In some embodiments, the VLP includes at least the M and E proteins. In some embodiments, the VLP includes at least the M, E, and S proteins. In some embodiments, the VLP includes the M, E, S, and N proteins. VLPs as described herein may be produced using any suitable method known in the art.

In some aspects, described herein are methods for producing a VLP in a mammalian cell. Mammalian cell-based systems for protein expression are known and described in the art. In general, a mammalian cell is transfected with a polynucleotide encoding a SARS-CoV-2 antigen or VLP and allowed to express said antigen or VLP from the polypeptide. The polynucleotides may be introduced into the mammalian cell using a construct or vector as described herein, for example, a MYXV vector. Mammalian cells suitable for protein expression are known in the art, including, but not limited to, Chinese Hamster Ovary (CHO), Madin-Darby Canine Kidney (MDCK), Vero, and HEK 293T cells.

Vaccine compositions including the SARS-CoV-2 antigens, VLPs, or myxoma viral vectors described herein are also provided. As used herein “vaccine” refers to a composition that includes an antigen. Vaccine may also include a biological preparation that improves immunity to a particular disease. A vaccine may typically contain an agent, referred to as an antigen, that resembles a disease-causing microorganism, in this case SARS-CoV-2, and the agent may often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters.

Vaccines may be prophylactic, e.g., to prevent or ameliorate the effects of a future infection by any natural or “wild” pathogen, or therapeutic, e.g., to treat the disease. Administration of the vaccine to a subject results in an immune response, generally against one or more specific diseases. The amount of a vaccine that is therapeutically effective may vary depending on the particular virus used, or the condition of the patient, and may be determined by a physician. The vaccine may be introduced directly into the subject by the subcutaneous, oral, oronasal, or intranasal routes of administration.

The vaccine compositions described herein also include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the SARS-CoV-2 antigens, VLPs, or RICs described herein.

The vaccine formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final vaccine formulation.

The remainder of the vaccine formulation may be an acceptable diluent, to 100%, including water. The vaccine formulation may also be formulated as part of a water-in-oil, or oil-in-water emulsion.

The vaccine formulation may be separated into vials or other suitable containers. The vaccine formulation herein described may then be packaged in individual or multi-dose ampoules, or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures. The lyophilized vaccine may be reconstituted by the end user, and administered to a patient.

The term “lyophilization” or “lyophilized,” as used herein, refers to freezing of a material at low temperature followed by dehydration by sublimation, usually under a high vacuum. Lyophilization is also known as freeze drying. Many techniques of freezing are known in the art of lyophilization such as tray-freezing, shelf-freezing, spray-freezing, shell-freezing and liquid nitrogen immersion. Each technique will result in a different rate of freezing. Shell-freezing may be automated or manual. For example, flasks can be automatically rotated by motor driven rollers in a refrigerated bath containing alcohol, acetone, liquid nitrogen, or any other appropriate fluid. A thin coating of product is evenly frozen around the inside “shell” of a flask, permitting a greater volume of material to be safely processed during each freeze drying run. Tray-freezing may be performed by, for example, placing the samples in lyophilizer, equilibrating 1 hr at a shelf temperature of 0° C., then cooling the shelves at 0.5° C./min to −40° C. Spray-freezing, for example, may be performed by spray-freezing into liquid, dropping by ˜20 μl droplets into liquid N2, spray-freezing into vapor over liquid, or by other techniques known in the art.

Methods of inducing an immune response in a subject are also provided. A vaccine composition as described herein and including a SARS-CoV-2 antigen, VLP, or myxoma viral vector as described herein is administered to subject to induce an immune response. Following administration, the immune response of the subject may be tested using methods known in the art.

To vaccinate a subject, a therapeutically effective amount of a vaccine composition described herein is administered to the subject. The therapeutically effective amount of vaccine may typically be one or more doses, preferably in the range of about 0.01-10 mL, most preferably 0.1-1 mL, containing 1-500 micrograms, most preferably 1-100 micrograms of vaccine formulation/dose. The therapeutically effective amount may also depend on the vaccination species. For example, for smaller animals such as mice, a preferred dosage may be about 0.01-1 mL of a 1-50 microgram solution of antigen. For a human patient, a preferred dosage may be about 0.1-1 mL of a 1-50 microgram solution of antigen. The therapeutically effective amount may also depend on other conditions including characteristics of the patient (age, body weight, gender, health condition, etc.), and others.

The term “administration,” as used herein, refers to the introduction of a substance, such as a vaccine, into a subject's body. The administration, e.g., parenteral administration, may include subcutaneous administration, intramuscular administration, transcutaneous administration, intradermal administration, intraperitoneal administration, intraocular administration, intranasal administration and intravenous administration.

The vaccine or the composition according to the invention may be administered to an individual according to methods known in the art. Such methods comprise application e.g. parenterally, such as through all routes of injection into or through the skin: e.g. intramuscular, intravenous, intraperitoneal, intradermal, mucosal, submucosal, or subcutaneous. Also, the vaccine may be applied by topical application as a drop, spray, gel or ointment to the mucosal epithelium of the eye, nose, mouth, anus, or vagina, or onto the epidermis of the outer skin at any part of the body.

Other possible routes of application are by spray, aerosol, or powder application through inhalation via the respiratory tract. In this last case, the particle size that is used will determine how deep the particles will penetrate into the respiratory tract.

Alternatively, application may be via the alimentary route, by combining with the food, feed or drinking water e.g. as a powder, a liquid, or tablet, or by administration directly into the mouth as a: liquid, a gel, a tablet, or a capsule, or to the anus as a suppository.

The present disclosure is generally applied to mammals, including but not limited to humans, cows, horses, sheep, pigs, goats, rabbits, dogs, cats, bats, mice and rats. In some embodiments, the present disclosure can be applied to birds. In certain embodiments, non-human mammals, such as mice and rats, may also be used for the purpose of demonstration. One may use the present invention for veterinary purpose. For example, one may wish to treat commercially important farm animals, such as cows, horses, pigs, rabbits, goats, sheep, and birds, such as chickens. One may also wish to treat companion animals, such as cats and dogs.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### Examples

Based on animal coronavirus vaccines and Dengue vaccine experiences, the current SARS-CoV-2 vaccines that express the Spike (S) protein alone have an increased risk for potentially severe disease in some people. Expressing the full VLP is likely to offer safer and more complete vaccination.

Myxoma has demonstrated ability to produce large quantities of multiple proteins in human or mouse leukocytes under the control of early/late viral promoters (the myxoma virus infection then aborts in non-cancerous human cells). Myxoma is a non-human pathogen, with no pre-existing antibodies in the human population, and will likely have fewer side effects than other live replicating vaccines. Myxoma also offers the possibility of intranasal delivery, like Flu-Mist, to provide mucosal immunity.

The following example describes the construction of a recombinant myxoma viruses (MYXV) that express SARS-CoV-2 proteins.

Design and Construction of a Recombinant vMyx-SARS2-S-dsRed:

vMyx-SARS2-S-dsRed was constructed by inserting a mammalian codon optimized SARS-CoV2 spike-expressing cassette between the M135 locus and M136 locus in the wild-type MYXV strain Lausanne (vMyx-Lau) genome. Spike expression is under the control of the poxvirus synthetic early-late promoter (sE/L). An additional cassette encoding a dsRed protein was inserted immediately downstream of the spike, and its expression was driven by a poxvirus p 11 late promoter (p 11). The dsRed can serve as a fluorescent marker for MYXV replication in vitro and in vivo, as MYXV infection can be monitored by live imaging of dsRed expression.

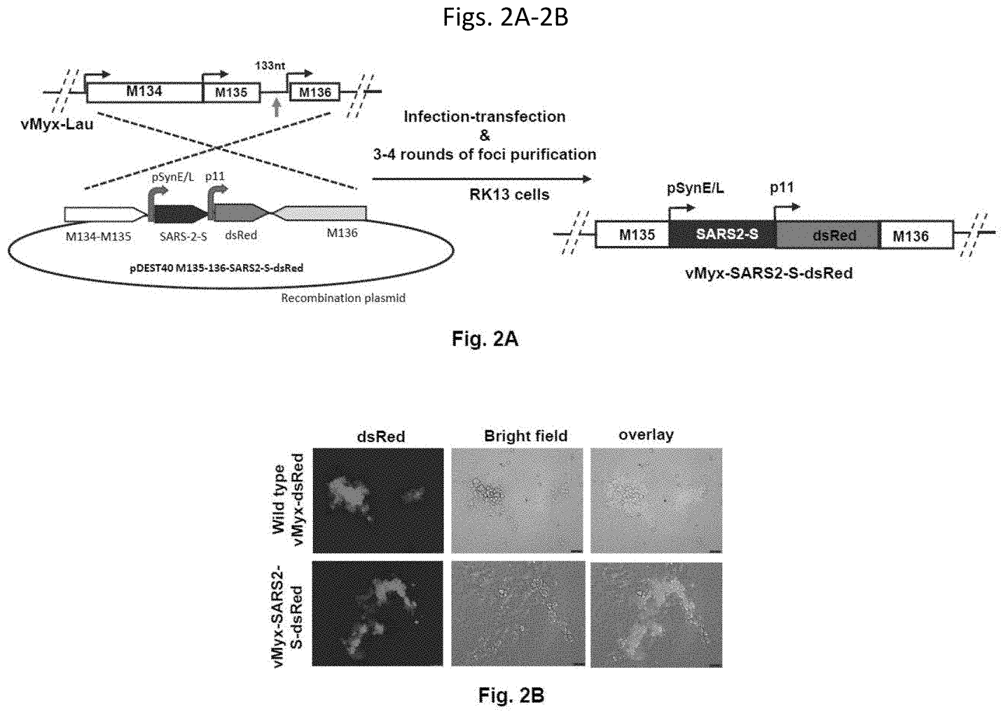

To create the vMyx-SARS2-S-dsRed construct, a recombinant plasmid was constructed using the Gateway System (ThermoFisher Scientific). FIG. 2A illustrates the design of the recombination plasmid and the location of the recombination cassette in the vMyx-SARS2-S-dsRed genome. To construct the recombinant plasmid, the upstream and downstream hybridizing sequences were amplified by PCR to generate entry clones by Gateway BP recombination with appropriate donor vectors. The final recombination plasmid pDEST40M135-M136-SARS2-S-dsRed was constructed by recombining three entry clones with the pDEST40 destination vector using a Gateway LR recombination reaction. The SARS2-S and dsRed expression cassettes were inserted into the MYXV genome by infecting RK13 cells with vMyx-Lau and then transfecting the recombination plasmid. Multiple rounds of foci purification were conducted to obtain purified stock of S-expressing recombinant virus (FIG. 2B).

SARS-CoV2 spike was amplified using the following primers:

Forward sequence: AttB4r-SARSCoV2-S-F:
(SEQ ID NO: 11)
GGGGACAACTTTTCTATACAAAGTTGCCAAAAATTGAAATTTTATTTTT
TTTTTTTGGAATATAAATAATGTTCGTGTTCCTGGTGCTTC
Reverse sequence: AttB3r-SARSCoV2-S-R:
(SEQ ID NO: 12)
GGGGACAACTTTATTATACAAAGTTGTTCAGGTGTAGTGCAGCTTCACG

Design and Construction of vMyx-SARS2-E-M-N-GFP:

vMyx-SARS2-E-M-N-GFP virus was constructed by inserting a mammalian codon optimized SARS-CoV2 E, M, and N-expressing cassette in between the myxoma virus M152 and M154 locus in the wild-type MYXV strain Lausanne (vMyx-Lau) genome. In this expression cassette (SEQ ID NO:13), which was synthesized by GenScript, the expression of each of the proteins is under the control of the poxvirus synthetic early-late promoter (sE/L). An additional cassette for an enhanced green fluorescent protein (eGFP) was inserted immediately downstream of the E-M-N cassette, and its expression was driven by a poxvirus sE/L promoter. The eGFP can serve as a fluorescent marker for MYXV replication in vitro and in vivo, as MYXV infection can be monitored by live imaging of GFP expression.

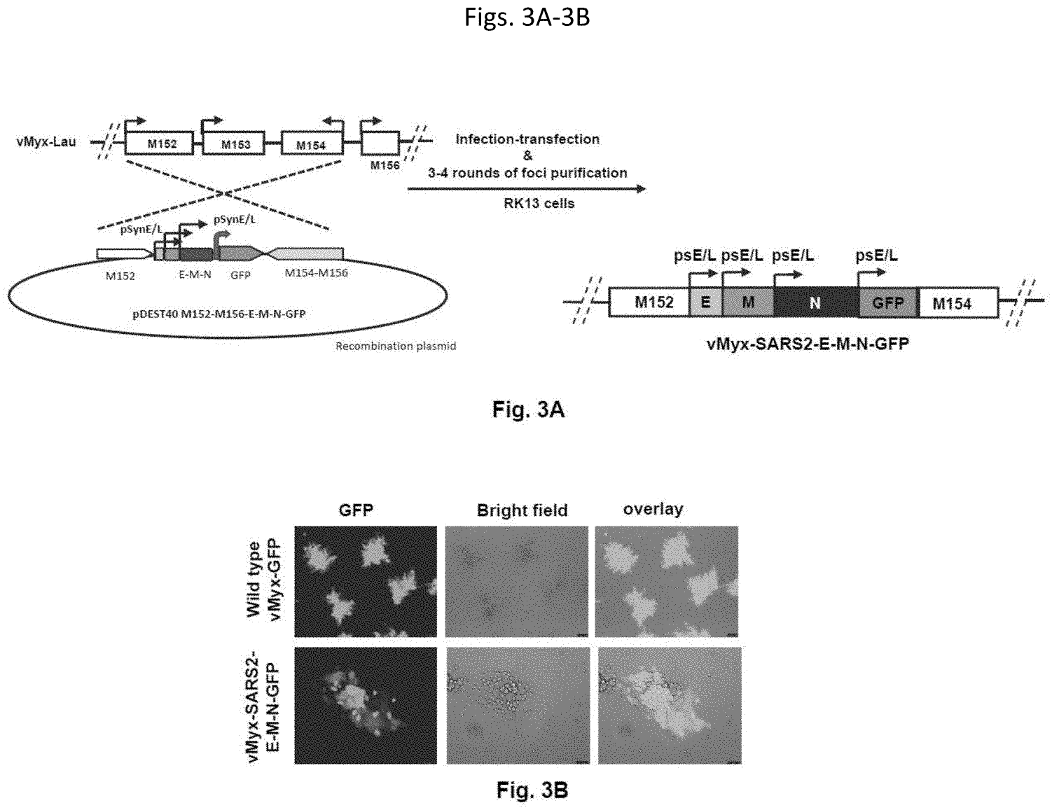

To create the vMyx-SARS2-E-M-N-GFP construct, a recombinant plasmid was constructed using the Gateway System (ThermoFisher Scientific). FIG. 3A illustrates the design of the recombination plasmid, and the location of the recombination cassette in the vMyx-SARS2-E-M-N-GFP genome. To construct the recombinant plasmid, the upstream and downstream hybridizing sequences of M152 and M154 were amplified by PCR to generate entry clones by Gateway BP recombination with appropriate donor vectors. The final recombination plasmid pDEST40M152-M154-SARS2-E-M-N-GFP was constructed by recombining three entry clones with the pDEST40 destination vector using Gateway LR recombination reaction. The SARS2-E-M-N and GFP expression cassettes were inserted into the MYXV genome by infecting RK13 cells with vMyx-Lau and then transfecting the recombination plasmid. Multiple rounds of foci purification were conducted to obtain purified stock of the E, M, and N-expressing recombinant virus (FIG. 3B).

Construction of vMyx-SARS2-S-E-M-N-GFP-dsRed:

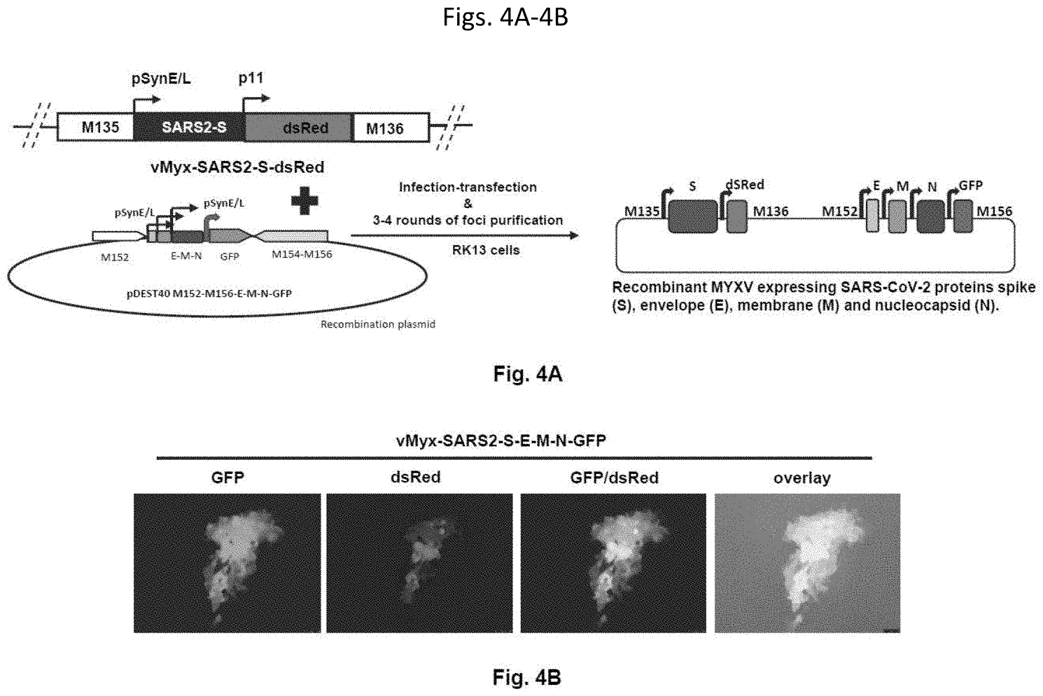

vMyx-SARS2-S-E-M-N-GFP-dsRed virus was constructed by inserting the SARS-CoV2 E, M, and N-expressing cassette in between the myxoma virus M152 and M154 locus in the S-expressing vMyx-SARS2-S-dsRed virus construct, as depicted in FIG. 4A. For this, RK13 cells were infected with vMyx-SARS2-S-dsRed virus and then transfected with the recombination plasmid pDEST40M152-M154-SARS2-E-M-N-GFP. Multiple rounds of foci purification were conducted to obtain purified stock of the S, E, M and N-expressing recombinant virus (FIG. 4A). The foci of the purified GFP and dsRed double positive S, E, M and N-expressing recombinant virus is shown after 48 h post infection (FIG. 4B).

Design and Construction of vMyx-SARS2-Hexa Pro S-dsRed:

We designed a modified spike protein, referred to herein as “Hexa Pro S” that includes a mutation in the furin cleavage site (rrar>gsas) and six proline mutations (i.e., F817P, A892P, A899P, A942P, KV986/7>PP) that collectively stabilize the pre-fusion S protein. The amino acid sequence of Hexa Pro S is provided as SEQ ID NO:14, and the nucleic acid sequence is provided as SEQ ID NO:15.

vMyx-SARS2-Hexa Pro S-dsRed was constructed by inserting a modified Hexa Pro Spike (Hexa Pro S)-expressing cassette in between the M135 and M136 locus in the wild-type MYXV strain Lausanne (vMyx-Lau) genome. Hexa Pro S expression is under the control of the poxvirus synthetic early-late promoter (sE/L). An additional cassette for a dsRed protein was inserted immediately downstream of the spike, and its expression was driven by a poxvirus p 11 late promoter (p 11). The dsRed can serve as a fluorescent marker for MYXV replication in vitro and in vivo, as MYXV infection can be monitored by live imaging of dsRed expression.

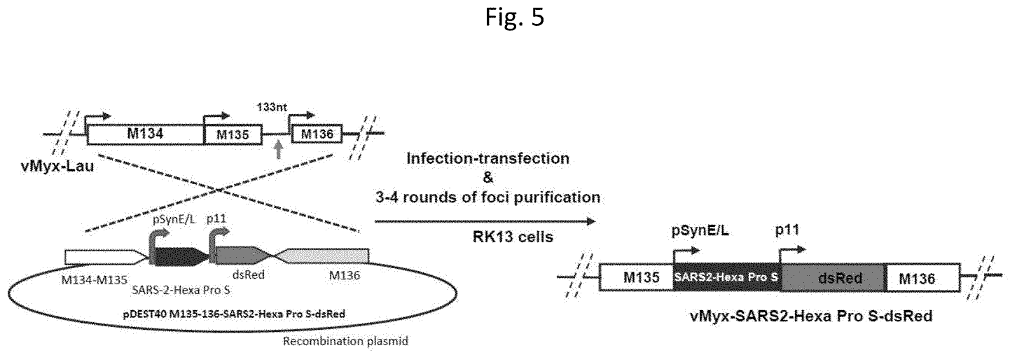

To create the vMyx-SARS2-Hexa Pro S-dsRed construct, a recombinant plasmid was constructed using the Gateway System (ThermoFisher Scientific). FIG. 5 illustrates the design of the recombination plasmid, and the location of the recombination cassette in the vMyx-SARS2-Hexa Pro S-dsRed genome. To construct the recombinant plasmid, the upstream and downstream hybridizing sequences were amplified by PCR to generate entry clones by Gateway BP recombination with appropriate donor vectors. The final recombination plasmid pDEST40M135-M136-SARS2-Hexa Pro S-dsRed was constructed by recombining three entry clones with the pDEST40 destination vector using Gateway LR recombination reaction. The SARS2-Hexa Pro S and dsRed expression cassettes were inserted into the MYXV genome by infecting RK13 cells with vMyx-Lau and then transfecting the recombination plasmid. Multiple rounds of foci purification were conducted to obtain purified stock of the S expressing recombinant virus.

Proper insertion was confirmed via PCR amplification of Hexa Pro S using the following primers:

Forward sequence: AttB4r-SARSCoV2-Hexa Pro S-F:
(SEQ ID NO: 16)
GGGGACAACTTTTCTATACAAAGTTGCCAAAAATTGAAATTTTATTTTTT
TTTTTTGGAATATAAATAATGTTTGTGTTTCTTGTTTTAT
Reverse sequence: AttB4r-SARSCoV2-Hexa Pro S-R:
(SEQ ID NO: 17)
GGGGACAACTTTATTATACAAAGTTGTTCATTATGTGTAATGTAATTTGA
CTCC

Construction of vMyx-SARS2-Hexa Pro S-E-M-N-GFP-dsRed:

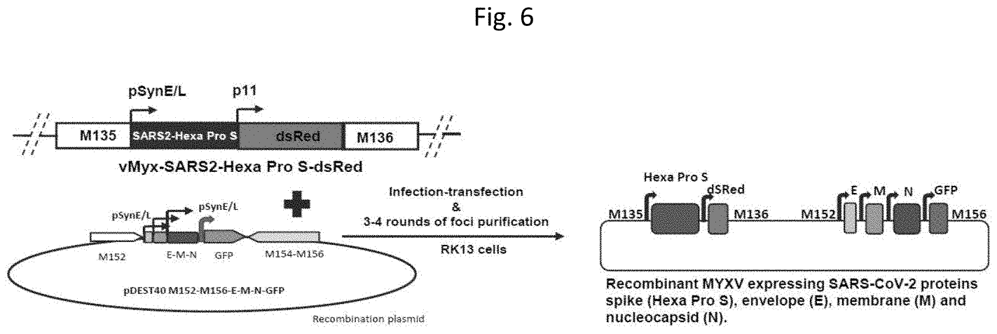

vMyx-SARS2-Hexa Pro S-E-M-N-GFP-dsRed virus was constructed by inserting the SARS-CoV2 E, M, and N-expressing cassette in between the myxoma virus M152 and M154 locus in the Hexa Pro S expressing vMyx-SARS2-Hexa Pro S-dsRed virus construct. For this, RK13 cells were infected with vMyx-SARS2-Hexa Pro S-dsRed virus and then transfected with the recombination plasmid pDEST40M152-M154-SARS2-E-M-N-GFP. Multiple rounds of foci purification were conducted to obtain purified stock of the Hexa Pro S, E, M and N-expressing recombinant virus (FIG. 6).

### Evaluation of Immunogenicity:

The recombinant viruses described herein may be used directly as a vaccine against SARS-CoV-2. Further, the recombinant viruses described herein may be used for infection of mammalian cells and used to produce virus-like particles (VLPs) that may be administered as a vaccine. To evaluate the immunogenicity of these vaccines, we will determine neutralizing activity of immunized sera. Mice will be immunized systemically and mucosally with the vaccines and serum and mucosal washes will be analyzed for induction of binding and neutralizing antibodies to SARS-CoV-2. Immunogens which induce neutralizing antibodies will be optimized by addition of adjuvants and/or prime-boost strategies to determine optimal formulations/regimens.

Systemic immunization. Balb/c mice will be immunized. Serum samples will be collected and analyzed for the presence of spike and RBD specific serum IgG by antigen-specific ELISAs. Serum IgG1 and IgG2a will also be analyzed to determine the type (Th1 vs Th2) of the immune response to individual immunogen/adjuvant combinations. Finally, serum samples will be assayed for neutralizing capacity using VSV pseudotyped with SARS-Coronavirus-2 S protein.

Mucosal immunization. Balb/c mice will be immunized intranasally in the absence or presence of synthetic CpG oligodeoxynucleotides (ODN) with selected recombinant MYXVs expressing VLPs or mammalian cell produced VLPs. If weak mucosal immune responses are observed following intranasal immunization only, we will try an intranasal priming/systemic boosting strategy which has been shown to be effective in augmenting sIgA production.

Neutralization assays. SARS-Coronavirus-2 neutralizing capacity of the serum and mucosal antibodies generated will be analyzed using VSV pseudotyped with SARS-Coronavirus-2 S protein. If weak mucosal responses are observed for VLP candidates, we will try other mucosal adjuvants (including LT mutant (R192G), saponin, and IVX-908 proteosome formulation (Protollin)). It is possible that antibodies against the RBD will not be sufficient to achieve the desired level of protection. If this is the case other neutralizing epitopes that have been identified in S will be incorporated into the VLPs and used in conjunction with the RBD VLPs or as part of a prime-boost regimen.

We claim:

1. A myxoma virus (MYXV) comprising a polynucleotide encoding a severe acute respiratory syndrome coronavirus 2 (SARS-CoV-2) antigen selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, SARS-CoV-2 nucleocapsid (N) protein (SEQ ID NO:5) or a sequence at least 95% identical thereto, and combinations thereof.

2. The MYXV of claim 1, wherein the MYXV comprises one or more polynucleotides encoding at least two SARS-CoV-2 antigens selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, and SARS-CoV-2 nucleocapsid (N) protein (SEQ ID NO:5) or a sequence at least 95% identical thereto.

3. The MYXV of claim 1, wherein the MYXV comprises one or more polynucleotides encoding at least three SARS-CoV-2 antigens selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, and SARS-CoV-2 nucleocapsid (N) protein (SEQ ID NO:5) or a sequence at least 95% identical thereto.

4. The MYXV of claim 1, wherein the MYXV comprises one or more polynucleotides encoding at least four SARS-CoV-2 antigens selected from the group consisting of SARS-CoV-2 spike (S) protein (SEQ ID NO:1) or a sequence at least 95% identical thereto, SARS-CoV-2 receptor binding domain (RBD) (SEQ ID NO:2) or a sequence at least 95% identical thereto, SARS-CoV-2 membrane (M) protein (SEQ ID NO:3) or a sequence at least 95% identical thereto, SARS-CoV-2 envelope (E) protein (SEQ ID NO:4) or a sequence at least 95% identical thereto, and SARS-CoV-2 nucleocapsid (N) protein (SEQ ID NO:5) or a sequence at least 95% identical thereto.

5. The MYXV of claim 1, wherein the polynucleotides encoding the antigens are one or more codon optimized polynucleotides selected from the group consisting of SEQ ID NO:18-21.

6. The MYXV of claim 1, wherein the polynucleotide encoding the modified SARS-CoV-2 S protein comprises SEQ ID NO:14 or a sequence at least 95% identical to SEQ ID NO: 14.

7. The MYXV of claim 1, wherein the MYXV comprises a modification at or adjacent to one or more genes associated with rabbit cell tropism.

8. The MYXV of claim 7, wherein the one or more genes associated with rabbit cell tropism are selected from the group consisting of M11L, M063, M135R, M136R, M153, M154, M-T2, M-T4, M-T5, and M-T7.

9. The MYXV of claim 8, wherein the MYXV comprises a partial or full deletion of the M153 and M154 genes.

10. The MYXV of claim 8, wherein the MYXV comprises a modification of the M153 and M154 genes that impairs the expression of the M153 and M154 genes.

11. The MYXV of claim 8, wherein the polynucleotide encoding the SARS-CoV-2 antigen replaces the M153 or the M154 gene in the MYXV genome.

12. The MYXV of claim 8, wherein the polynucleotide encoding the SARS-CoV-2 antigen is inserted between the M153 gene and the M154 gene within the MYXV genome.

13. A vaccine composition comprising the MYXV of claim 1 and a pharmaceutically acceptable carrier or adjuvant.

14. (canceled)

15. A method for producing a SARS-CoV-2 virus-like particle (VLP) comprising
transfecting a mammalian cell with the MYXV of claim 1; and
extracting the SARS-CoV-2 VLP from the mammalian cell.

16. The method of claim 15, wherein the mammalian cell is selected from the group consisting of Chinese Hamster Ovary (CHO), Madin-Darby Canine Kidney (MDCK), Vero, and HEK 293T.

17. A VLP produced by the method of claim 15.

18. A method for inducing an immune response in a subject comprising administering an effective amount of the vaccine composition of claim 13 to the subject.

19. A method of inducing an immune response in a subject comprising administering an effective amount of the VLP of claim 17 to the subject.

20. (canceled)

21. (canceled)

22. The method of claim 18, wherein the vaccine composition is administered by injection or intranasally.

23. (canceled)

24. The method of claim 18, wherein the vaccine composition is administered in at least two doses.


SEQUENCE LISTING (21 sequences)

SEQ ID NO: 1 - protein, 1273 aa, SARS-CoV-2
  MISC_FEATURE (1)..(1273): Spike (S) protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 193 aa, SARS-CoV-2
  MISC_FEATURE (1)..(193): Receptor binding domain (RBD) of spike protein (amino acids 318-510 of SEQ ID NO1)
FRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 3 - protein, 222 aa, SARS-CoV-2
  MISC_FEATURE (1)..(222): Membrane (M) protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 75 aa, SARS-CoV-2
  MISC_FEATURE (1)..(75): Envelope (E) protein
MYSFVSEETGTLIVNSVLLFLAFVVFLLVTLAILTALRLCAYCCNIVNVSLVKPSFYVYSRVKNLNSSRVPDLLV

SEQ ID NO: 5 - protein, 419 aa, SARS-CoV-2
  MISC_FEATURE (1)..(419): Nucleocapsid (N) protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - DNA, 11 bp, Artificial Sequence
  Synthetic- translation enhancing element (TEE)
aaaactgctaa

SEQ ID NO: 7 - DNA, 37 bp, Artificial Sequence
  Synthetic- translation enhancing element (TEE)
catattgaagagacagagtgatatataaaactgctaa

SEQ ID NO: 8 - DNA, 42 bp, Artificial Sequence
  Synthetic- translation enhancing element (TEE)
agaaccatattgaagagacagagtgatatataaaactgctaa

SEQ ID NO: 9 - DNA, 90 bp, Artificial Sequence
  Synthetic- translation enhancing element (TEE)
agaaccatattgaagagacagagtgatatataaaactgctaactcaagcagcacaagaattaaatgaataccaagaaaatacttggccag

SEQ ID NO: 10 - DNA, 26 bp, Artificial Sequence
  Synthetic- viral synthetic late promoter (SLP)
catattgaagagacagagtgatatat

SEQ ID NO: 11 - DNA, 90 bp, Artificial Sequence
  Synthetic- Primer for amplifying SARS-CoV2 spike
ggggacaacttttctatacaaagttgccaaaaattgaaattttattttttttttttggaatataaataatgttcgtgttcctggtgcttc

SEQ ID NO: 12 - DNA, 49 bp, Artificial Sequence
  Synthetic- Primer for amplifying SARS-CoV2 spike
ggggacaactttattatacaaagttgttcaggtgtagtgcagcttcacg

SEQ ID NO: 13 - DNA, 2332 bp, Artificial Sequence
  Synthetic - Nucleotide sequences of E-M-N under sE/L promoter
ggggacaacttttctatacaaagttgccaaaaattgaaattttattttttttttttggaatataaataatgtactcttttgtgagtgaagagacaggcaccctgatcgtgaatagcgtgctgttgtttctggccttcgtggtgttcctgctggtgaccctggctatcctgaccgccctgagactgtgcgcctactgctgcaacatcgtgaacgtgtccctggtcaagcccagcttctacgtgtacagcagagtgaagaacctgaacagcagccgggtgcccgacctgctggtgtgaaaaaattgaaattttattttttttttttggaatataaataatggctgatagcaacggcacaatcaccgtggaagagctgaagaagctgcttgagcagtggaacctggtgattggatttctgtttctgacctggatctgcctgctgcagttcgcatacgccaaccggaacagattcctgtacatcatcaagctgatcttcctgtggctgctgtggcctgtgaccctggcctgcttcgtgctggccgccgtgtaccggatcaactggatcaccggcggaatcgccatcgccatggcctgtctggtgggcctgatgtggctgtcttatttcatcgcctcttttgccatcgccatggcctgtctggtgggcctgatgtggctgtcttatttcatcgcctcttttaatgtgccactgcacggcaccatcctgacaagacctctgcttgaaagcgagctggtgatcggcgccgtgatcctgcggggccacctgagaatcgccggacaccacctgggcagatgcgacatcaaggacctgcctaaggaaatcaccgtggccaccagcagaacactgagctactacaagctcggcgcttctcagagagtggccggcgacagcggcttcgccgcttacagccggtacagaatcggcaactacaagctgaacaccgaccacagctccagcagcgacaacatcgccctgctggtccagtgaaaaaattgaaattttattttttttttttggaatataaataatgtctgacaacggccctcaaaaccagcggaacgcccctcggataacatttggaggacccagcgacagcacaggctccaaccagaacggcgagagaagcggcgccagatctaaacagaggcggcctcagggcctgccaaacaacaccgccagctggttcaccgccctgacccaacacggcaaagaggacctcaagttccctagaggacagggcgtgcccatcaacacgaacagcagccccgacgaccagatcggctactacagacgggccacaagacggatcagaggcggcgacggcaagatgaaagacctgagcccaagatggtacttctactacctgggcaccggccccgaggccggcctgccctacggcgccaataaggacggcatcatctgggtcgccacagagggcgcactgaacacccccaaggaccacatcggaactagaaaccccgccaacaacgccgcaatcgtgctgcaactgccccaaggcacgaccctgcctaagggcttctacgccgaaggctctagaggcggcagccaggcttcttctcggagctcctcccggagccggaacagctctagaaattctacacctggcagcagcagaggcaccagccctgctagaatggccggcaacggcggagatgccgccctggccctgctgctgcttgatagactgaatcagctggaaagcaaaatgagcggaaaaggccagcaacagcaaggacaaacagtgacaaagaaaagcgccgctgaagcctctaagaaacctagacagaaaagaaccgccaccaaggcctacaacgtgacccaggcctttggcagaaggggacctgagcagacccagggaaatttcggcgatcaggagctgatccggcagggcaccgactacaagcactggcctcagatcgcccagttcgcccctagcgccagcgcttttttcggcatgagccggatcggcatggaagtgacccctagcggcacctggctgacatacaccggcgccatcaagctggacgataaggatcctaacttcaaggatcaggtgatcctgctgaacaagcatatcgacgcctataagaccttcccccccaccgagcccaagaaggacaagaagaaaaaggccgacgagacacaagctctgcctcagagacagaagaaacagcagaccgtgaccctgctgcctgccgccgacctggacgacttcagcaagcagctgcagcagagcatgagctctgctgatagcacccaggcctgaacaactttgtataataaagttgtcccc

SEQ ID NO: 14 - protein, 1273 aa, Artificial Sequence
  Synthetic- amino acid sequence of modified spike protein “Hexa Pro S”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3822 bp, Artificial Sequence
  Synthetic- nucleic acid sequence of modified spike protein “Hexa Pro S”
atgtttgtgtttcttgttttattaccattagtctctagtcagtgtgttaatttaacaaccagaactcaattaccccctgcatacactaattctttcacaagaggtgtttattaccctgacaaagttttcagatcctcagttttacattcaactcaggacttattcttacctttcttttccaatgttacttggttccatgctatacatgtctctgggaccaatggtactaagaggtttgataaccctgtcttaccatttaatgatggtgtttattttgcttccactgagaagtctaacataataagaggctggatttttggtactactttagattcgaagacccagtccttattaattgttaataacgctactaatgttgttattaaagtctgtgaatttcaattttgtaatgatccatttttaggtgtttattaccacaaaaacaacaaaagttggatggaaagtgagttcagagtttattctagtgcgaataattgcacttttgaatatgtctctcagccttttttaatggaccttgaaggaaaacagggtaatttcaaaaatttaagagaatttgtgtttaagaatattgatggttattttaaaatatattctaagcacacgcctattaatttagtgagagatctccctcagggtttttcggctttagaaccattggtagatttaccaataggtattaacatcactagatttcaaactttattagctttacatagaagttatttaactcctggtgattcttcttcaggttggacagctggtgctgcagcttattatgtgggttatttacaacctagaactttcttattaaaatataatgaaaatggaaccattacagatgctgtagactgtgcattagaccctttatcagaaacaaagtgtacgttaaaatccttcactgtagaaaaaggaatctatcaaacttctaactttagagtccaaccaacagaatctattgttagatttcctaatattacaaacttatgcccttttggtgaagtttttaacgccaccagatttgcatctgtttatgcttggaacagaaagagaatcagcaactgtgttgctgattattctgtcttatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatttagattctaaggttggtggtaattataattacttatatagattatttagaaagtctaatttaaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtattatcttttgaacttttacatgcaccagcaactgtttgtggacctaaaaagtctactaatttagttaaaaacaaatgtgtcaatttcaacttcaatggtttaacaggcacaggtgttttaactgagtctaacaaaaagtttctgcctttccaacaatttggcagagacattgctgacactactgatgctgtcagagatccacagacattagagattttagacattacaccatgttcttttggtggtgtcagtgttataacaccaggaacaaatacttctaaccaggttgctgttttatatcaggatgttaactgcacagaagtccctgttgctattcatgcagatcaattaactcctacttggagagtttattctacaggttctaatgtttttcaaacaagagcaggctgtttaataggggctgaacatgtcaacaactcatatgagtgtgacatacccattggtgcaggtatatgcgctagttatcagactcagactaattctcctgggagcgcaagtagtgtagctagtcaatccatcattgcctacactatgtcattaggtgcagaaaattcagttgcttactctaataactctattgccatacccacaaattttactattagtgttaccacagaaattttaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagagatcaccaattgaagatttacttttcaacaaagtgacattagcagatgctggcttcatcaaacaatatggtgattgcttaggtgatattgctgctagagacttaatttgtgcacaaaagtttaacggcttaactgttttaccacctttattaacagatgaaatgattgctcaatacacttctgcattattagcgggtacaatcacttctggttggacctttggtgcaggtccggcattacaaataccatttccgatgcaaatggcttatagatttaatggtattggagttacacagaatgttttatatgagaaccaaaaattaattgccaaccaatttaatagtgctattggcaaaattcaagactcattatcttccacaccaagtgcattaggaaaattacaagatgtggtcaaccaaaatgcacaagctttaaacacgttagttaaacaattaagctccaattttggtgcaatttcaagtgttttaaatgatatcttatcaagattagacccaccagaggctgaagtgcaaattgatagattaatcacaggcagattacaaagtttacagacatatgtgactcaacaattaattagagctgcagaaatcagagcttctgctaatttagctgctactaaaatgtcagagtgtgtattaggacaatcaaaaagagttgatttctgtggaaagggctatcatttaatgtccttccctcagtcagcacctcatggtgtagtcttcttacatgtgacttatgtccctgcacaagaaaagaacttcacaactgctcctgccatttgtcatgatggaaaagcacactttcctagagaaggtgtctttgtttcaaatggcacacactggtttgtaacacaaagaaatttctatgaaccacaaatcattactacagacaacacatttgtgtctggtaactgtgatgttgtaataggaattgtcaacaacacagtttatgatcctttacaacctgaattagactcattcaaggaggagttagataaatattttaagaatcatacatcaccagatgttgatttaggtgacatctctggcattaatgcttcagttgtaaacattcaaaaagaaattgacagattaaatgaggttgccaagaatttaaatgaatctttaatcgatttacaagaattaggaaagtatgagcagtatataaaatggccatggtacatttggttaggttttatagctggcttaattgccatagtaatggtgacaattatgttatgctgtatgaccagttgctgtagttgtttaaagggctgttgttcttgtggatcctgctgcaaatttgatgaagacgactctgagccagtgttaaaaggagtcaaattacattacacataa

SEQ ID NO: 16 - DNA, 90 bp, Artificial Sequence
  Synthetic- Primer for amplifying SARSCoV2-Hexa Pro spike
ggggacaacttttctatacaaagttgccaaaaattgaaattttattttttttttttggaatataaataatgtttgtgtttcttgttttat

SEQ ID NO: 17 - DNA, 54 bp, Artificial Sequence
  Synthetic- Primer for amplifying SARSCoV2-Hexa Pro spike
ggggacaactttattatacaaagttgttcattatgtgtaatgtaatttgactcc

SEQ ID NO: 18 - DNA, 3822 bp, Artificial Sequence
  Synthetic- Codon optimized sequence encoding S protein
atgttcgtgttcctggtgcttctgcctctggttagcagccagtgcgtgaacctgaccacccggacccagctgccacctgcttacacaaacagcttcaccagaggcgtgtactaccccgataaggtgttcagaagcagcgtgctgcacagcacacaggacctgttcctgcctttcttcagcaacgtgacatggttccacgccatccacgtgtccggcacaaacggcacaaagcggttcgataaccctgtgctacctttcaatgacggcgtgtattttgcctctaccgagaagtctaacatcattcggggctggatcttcggcaccaccctggacagcaaaacccagagcctgctgatcgtgaacaacgccactaacgtggtgatcaaagtttgtgaatttcagttctgcaacgacccgtttctgggcgtttactaccacaagaacaacaagagctggatggaatccgagttcagagtgtactcgtctgccaacaactgtaccttcgagtacgtgtcccagcccttcctgatggacctggaaggcaagcagggcaacttcaagaatctgagagagtttgtgttcaagaatatcgacggctacttcaagatctacagcaagcacaccccgatcaacctggtgcgagatctgccccaaggcttcagcgccctggaaccactggttgacctgcccatcggaatcaacattacgcggttccaaacgctgctggccctgcatagaagctacctgacacctggagattccagctccggctggaccgccggcgccgctgcctactacgtgggctacctgcagcctagaaccttcctgctgaaatataatgagaacggcaccatcacagacgccgtggactgtgccctggatcccttgtctgagacaaagtgcacactgaagagcttcacagtggaaaaaggcatctaccaaacatccaacttccgggtccagcctacagaaagcatcgtgcggtttcccaacatcaccaatctgtgtccttttggcgaggttttcaatgcaacaagattcgcttctgtctacgcctggaatcggaagagaatctctaactgcgtggccgattacagcgttctgtacaacagcgccagcttttccacctttaagtgctacggcgtcagtcctaccaagctgaacgacttgtgcttcaccaacgtgtacgccgactccttcgtgatccggggcgacgaggtgcggcagatcgcccctgggcagaccggcaagatcgcggattacaactacaagctgcctgatgattttaccggatgcgtgattgcttggaacagcaacaacctggactctaaggttggcggaaactacaactacctgtatagactgtttagaaagtccaacctgaagcccttcgaaagagacatcagcacagagatctaccaggccggcagtaccccatgtaatggtgttgagggcttcaactgctacttccctctgcagtcttacggcttccagcctacaaacggcgtgggctaccagccctacagagtggtggtcctcagcttcgagctcctgcacgctcctgccaccgtctgcggtcctaagaagagcacaaacctggtgaagaataagtgcgtgaatttcaactttaacggcctgaccggcaccggagtgctgacggaaagcaacaaaaagttcctgcccttccagcaatttggaagagatatcgctgacaccacagacgctgtgcgggatccgcagactctggaaatcctggacatcaccccctgttctttcggaggcgtgagcgtgatcacgcctggcaccaacaccagcaaccaggtggccgtgctgtaccaggacgttaattgcaccgaggtgccagttgccatccacgccgatcagctgaccccaacatggcgggtgtattctacaggtagcaacgtgttccagacccgggccggctgcctgatcggcgccgagcacgtcaataatagctacgagtgcgacatccccattggagctggcatctgcgccagctaccagacgcaaacaaacagccctcgtagagcccggagcgtggccagtcagagtatcatcgcttacaccatgagcctgggcgccgaaaacagcgtggcctattctaacaatagcatcgccatccctaccaacttcaccatctctgtgacaaccgagattctgcctgtatccatgacaaagaccagtgtcgactgcactatgtacatctgcggcgatagcaccgaatgttctaacctcctgttgcagtatggaagcttttgtactcagctcaacagagccctgacaggcattgctgtggaacaggataagaacacccaggaggtgttcgcccaagtgaagcagatctataagacacctcctatcaaggacttcggcggcttcaactttagccagatcctgcctgacccgagcaagcctagcaaaaggtccttcatcgaggaccttctcttcaacaaggtgaccttggccgacgccggatttatcaagcagtacggcgactgcctgggagatatcgccgctagagatctgatttgtgctcaaaagttcaatggcctgaccgtgttaccgcctctgttgaccgatgagatgatcgcccagtacaccagcgccctgctggccggaacaatcaccagcggctggacattcggtgccggcgccgccctgcaaatccccttcgccatgcagatggcctaccggttcaatggtatcggcgtgacacaaaacgtgctgtacgagaaccagaaactgatcgccaaccagttcaacagcgccatcggaaagatccaggattctctgtcttctaccgccagtgccctgggcaagctgcaagacgtggtcaaccagaacgcccaggccctgaacacactggtgaaacagctgagctccaacttcggcgccatcagctccgtactgaatgacatcctgtcaagactggataaggtggaagccgaggtgcagattgatagactgatcaccggcagactgcagagcctgcagacatacgtgacccagcagctgatccgggctgctgagatcagagctagtgctaatctcgccgctacaaagatgagcgaatgcgtgctgggacagagcaagagagtggacttctgcggcaaggggtatcacctgatgtccttcccacagagcgcacctcacggcgtggtgtttttacatgtgacctacgtgcctgcccaggagaagaactttaccacggcccctgctatctgccacgatggcaaagcccacttccccagagagggcgtgttcgtaagcaacggcacacactggtttgtgacccagagaaacttctacgagccccagatcatcaccaccgacaacaccttcgtgtctggcaattgtgatgtggtgatcggcatcgtgaacaacaccgtgtacgaccctctgcagcccgagctggacagcttcaaagaggaactggacaagtacttcaagaaccacaccagccctgatgtggacctcggagacatcagcggcatcaacgcctccgtggtgaacatccagaaggaaattgacagactgaatgaggtggccaagaaccttaatgagagcctgatcgacctccaggagctgggcaagtacgagcagtacatcaagtggccctggtacatctggctggggttcattgcaggactgatcgccatcgtgatggtgaccatcatgctgtgctgcatgacctcttgttgtagctgcctgaagggctgctgctcctgcggcagctgttgcaagttcgacgaggatgacagcgagcctgtgctgaaaggcgtgaagctgcactacacctga

SEQ ID NO: 19 - DNA, 228 bp, Artificial Sequence
  Synthetic- Codon optimized sequence encoding E protein
atgtactcttttgtgagtgaagagacaggcaccctgatcgtgaatagcgtgctgttgtttctggccttcgtggtgttcctgctggtgaccctggctatcctgaccgccctgagactgtgcgcctactgctgcaacatcgtgaacgtgtccctggtcaagcccagcttctacgtgtacagcagagtgaagaacctgaacagcagccgggtgcccgacctgctggtgtga

SEQ ID NO: 20 - DNA, 669 bp, Artificial Sequence
  Synthetic- Codon optimized sequence encoding M protein
atggctgatagcaacggcacaatcaccgtggaagagctgaagaagctgcttgagcagtggaacctggtgattggatttctgtttctgacctggatctgcctgctgcagttcgcatacgccaaccggaacagattcctgtacatcatcaagctgatcttcctgtggctgctgtggcctgtgaccctggcctgcttcgtgctggccgccgtgtaccggatcaactggatcaccggcggaatcgccatcgccatggcctgtctggtgggcctgatgtggctgtcttatttcatcgcctcttttagactgttcgccagaaccagaagcatgtggtccttcaaccccgagacaaacatcctgcttaatgtgccactgcacggcaccatcctgacaagacctctgcttgaaagcgagctggtgatcggcgccgtgatcctgcggggccacctgagaatcgccggacaccacctgggcagatgcgacatcaaggacctgcctaaggaaatcaccgtggccaccagcagaacactgagctactacaagctcggcgcttctcagagagtggccggcgacagcggcttcgccgcttacagccggtacagaatcggcaactacaagctgaacaccgaccacagctccagcagcgacaacatcgccctgctggtccagtga

SEQ ID NO: 21 - DNA, 1260 bp, Artificial Sequence
  Synthetic- Codon optimized sequence encoding N protein
atgtctgacaacggccctcaaaaccagcggaacgcccctcggataacatttggaggacccagcgacagcacaggctccaaccagaacggcgagagaagcggcgccagatctaaacagaggcggcctcagggcctgccaaacaacaccgccagctggttcaccgccctgacccaacacggcaaagaggacctcaagttccctagaggacagggcgtgcccatcaacacgaacagcagccccgacgaccagatcggctactacagacgggccacaagacggatcagaggcggcgacggcaagatgaaagacctgagcccaagatggtacttctactacctgggcaccggccccgaggccggcctgccctacggcgccaataaggacggcatcatctgggtcgccacagagggcgcactgaacacccccaaggaccacatcggaactagaaaccccgccaacaacgccgcaatcgtgctgcaactgccccaaggcacgaccctgcctaagggcttctacgccgaaggctctagaggcggcagccaggcttcttctcggagctcctcccggagccggaacagctctagaaattctacacctggcagcagcagaggcaccagccctgctagaatggccggcaacggcggagatgccgccctggccctgctgctgcttgatagactgaatcagctggaaagcaaaatgagcggaaaaggccagcaacagcaaggacaaacagtgacaaagaaaagcgccgctgaagcctctaagaaacctagacagaaaagaaccgccaccaaggcctacaacgtgacccaggcctttggcagaaggggacctgagcagacccagggaaatttcggcgatcaggagctgatccggcagggcaccgactacaagcactggcctcagatcgcccagttcgcccctagcgccagcgcttttttcggcatgagccggatcggcatggaagtgacccctagcggcacctggctgacatacaccggcgccatcaagctggacgataaggatcctaacttcaaggatcaggtgatcctgctgaacaagcatatcgacgcctataagaccttcccccccaccgagcccaagaaggacaagaagaaaaaggccgacgagacacaagctctgcctcagagacagaagaaacagcagaccgtgaccctgctgcctgccgccgacctggacgacttcagcaagcagctgcagcagagcatgagctctgctgatagcacccaggcctga